(19) United States
(12) Patent Application Publication
(10) Pub. No.: US 2021/0198682 A1
(43) Pub. Date: Jul. 1, 2021

(54) APPLICATION OF SDG40 GENE OR ENCODED PROTEIN THEREOF

(71) Applicant: CAS CENTER FOR EXCELLENCE IN MOLECULAR PLANT SCIENCES, Shanghai (CN)

(72) Inventors: Xinguang ZHU, Shanghai (CN); Mingnan QU, Shanghai (CN); Genyun CHEN, Shanghai (CN); Chengcai CHU, Shanghai (CN)

(21) Appl. No.: 17/057,813

(22) PCT Filed: May 22, 2019
(86) PCT No.: PCT/CN2019/087976

§ 371 Date: Nov. 23, 2020

(30) Foreign Application Priority Data
May 24, 2018 (CN) 201810509900.7

(51) Int. Cl. C12N 15/82; C07K 14/415

(52) U.S. Cl. CPC C12N 15/8218; C12N 15/8269; C12N 15/8261; C07K 14/415

(57) ABSTRACT

Disclosed is an application of SDG40 gene or an encoded protein thereof. Specifically, when the expression of SDG40 gene or an encoded protein thereof is inhibited, agronomic traits of crops can be significantly improved, which include: (i) improved low light utilization efficiency (A low); (ii) increased biomass; (iii) increased number of tillers; (iv) increased yield per plant; and/or (v) increased plant height. In addition, it was also found that a mutation of the promoter region of the SDG gene from C to T and/or a mutation to from A to C can also significantly improve the low light utilization efficiency (A low) of crops.

### TECHNICAL FIELD

The present invention relates to the field of agronomy, in particular, to an application of SDG40 gene or encoded protein thereof.

### BACKGROUND

Photosynthesis is the most important biological reaction on earth, regulating the global balance of carbon dioxide and oxygen. The economic output of crops is mainly determined by photosynthetic efficiency. Rice is the largest food crop in my country, most of the leaves located in the lower part of the rice canopy are in a low light environment, especially in regionalized atmospheric visibility reduction (such as haze and other weather), which can seriously affect the economic output of rice (newsxinhua). Therefore, improving the relationship of light energy utilization efficiency in rice under low light is of great significance for improving my country's food production and food security strategic guarantee.

RUBISCO (ribulose-1,5 bisphosphate carboxylase/oxgenase) is an important regulatory enzyme in plant photosynthetic carbon metabolism, which can account for 50% of the total protein content of leaves. However, the catalytic efficiency of RUBISCO is low. At the same time, RUBISCO is oxygen-activated, consumes oxygen, and reduces photosynthetic efficiency. It has been widely reported that RUBISCO activity and photosynthetic efficiency have been regulated and improved through a series of genetics and molecular biology methods, but the progress is slow.

In recent years, the effects of non-histone methyltransferases (such as p53) that affect protein post-translational modifications (PTMs) in animal cancerous cells have been reported, wherein in the SETDOMAIN gene family, one class (CLASS IIB) can encode non-histone protein (mainly chloroplast protein) methyltransferase. In rice, there are 5 members in total. Among them, the large subunit methyltransferase (LSMT1) can catalyze the transfer of the methyl group of S-methionine (SAM) to the lysine 14 residue of Rubisco and the lysine 395 residue of fructose 1,6 diphosphate (FBA); however, there is no relevant obvious biological function.

Therefore, the identification of new types of chloroplast protein methyltransferases and their biological functions is essential to improve the efficiency of photosynthetic carbon metabolism and economic yield.

### SUMMARY OF THE INVENTION

The purpose of the present invention is to provide a new type of chloroplast protein methyltransferase whose biological function is essential for improving the efficiency of photosynthetic carbon metabolism and economic yield.

In a first aspect of the present invention, it provides a use of an inhibitor of SDG40 gene or encoded protein thereof for the regulation of an agronomic trait of a plant or preparing a preparation or composition for the regulation of an agronomic trait of a plant, wherein the agronomic trait of the plant is selected from one or more of the following groups:

(i) low light utilization efficiency (A_{low});

(ii) biomass;

(iii) the number of tillers;

(iv) yield per plant;

(v) plant height.

In another preferred embodiment, the “the regulation of an agronomic trait of a plant” comprises:

(i) the improvement of low light utilization efficiency (A_{low}); and/or

(ii) the increasement of biomass; and/or

(iii) the increasement of the number of tillers; and/or

(iv) the increasement of the yield per plant; and/or

(v) the increasement of plant height.

In another preferred embodiment, the composition comprises an agricultural composition.

In another preferred embodiment, the inhibitor is selected from the group consisting of: antisense nucleic acid, antibody, small molecule compound, Crispr reagent, siRNA, shRNA, miRNA, small molecule ligand, and a combination thereof.

In another preferred embodiment, the plant is selected from the group consisting of: Salicaceae, Moraceae, Myrtaceae, Lycopodiaceae, Selaginellaceae, Ginkgoaceae, Pinaceae, Cycadaceae, Araceae, Ranunculaceae, Platanaceae, Ulmaceae, Juglandaceae, Betulaceae, Actinidiaceae, Malvaceae, Sterculiaceae, Tiliaceae, Tamaricaceae, Rosaceae, Crassulaceae, Caesalpinaceae, Fabaceae, Punicaceae, Nyssaceae, Cornaceae, Alangiaceae, Celastraceae, Aquifoliaceae, Buxaceae, Euphorbiaceae, Pandaceae, Rhamnaceae, Vitaceae, Anacardiaceae, Burseraceae, Campanulaceae, Rhizophoraceae, Sandalaceae, Oleaceae, Scrophulariaceae, Gramineae, Pandanaceae, Sparganiaceae, Aponogeonaceae, Potamogetonaceae, Najadaceae, Scheuchzeriaceae, Alismataceae, Butomaceae, Hydrocharitaceae, Triuridaceae, Cyperaceae, Palmae (Arecaceae), Araceae, Lemnaceae, Flagellariaceae, Restionaceae, Centrolepidaceae, Xyridaceae, Eriocaulaceae, Bromeliaceae, Commelinaceae, Pontederiaceae, Philydraceae, Juncaceae, Stemonaceae, Liliaceae, Amaryllidaceae, Taccaceae, Dioscoreaceae, Iridaceae, Musaceae, Zingiberaceae, annaceae, Marantaceae, Burmanniaceae, Chenopodiaceae or Orchidaceae.

In another preferred embodiment, the Gramineous plant is selected from (but is not limited to): wheat, rice, barley, oats, and rye;

the Cruciferous plant is selected from (but is not limited to): oilseed rape, Chinese cabbage and other vegetables;

the Malvaceae plant is selected from (but is not limited to): cotton, Chinese hibiscus, hibiscus;

the Leguminous plant is selected from (but is not limited to): soybean, alfalfa, etc.;

the Solanaceae plant includes but is not limited to: tobacco, tomato, pepper, etc.;

the Cucurbitaceous plant includes but is not limited to: pumpkin, watermelon, cucumber, etc.;

the Rosaceous plant includes but is not limited to: apple, peach, plum, Chinese flowering crabapple, etc.;

the Chenopodiaceae plant is selected from (but is not limited to): beet;

the Compositae plant includes but is not limited to: sunflower, lettuce, asparagus lettuce, sweet wormwood, jerusalem artichoke, stevia rebaudiana, etc.;

the Salicaceae plant includes but is not limited to: poplar, willow, etc.;

the Myrtaceae plant includes but is not limited to: eucalyptus, girofle, myrtle, etc.;

the Euphorbiaceae plant includes but is not limited to: rubber tree, cassava, castor-oil plant, etc.;

the Papilionaceae plant includes but is not limited to: peanut, pea, Astragalus membranaceus, etc.

In another preferred embodiment, the plant is selected from the group consisting of: rice, wheat, sorghum, corn, green bristlegrass, tobacco, Arabidopsis, and a combination thereof.

In another preferred embodiment, the rice is selected from the group consisting of: indica type rice, japonica rice, and a combination thereof.

In another preferred embodiment, the SDG40 gene includes a cDNA sequence, a genomic sequence, and a combination thereof.

In another preferred embodiment, the SDG40 gene is from one or more crops from the following group: Gramineae, Solanaceae, and Cruciferae.

In another preferred embodiment, the SDG40 gene is from one or more crops selected from the group consisting of: rice, wheat, tobacco, Arabidopsis, corn, and a combination thereof.

In another preferred embodiment, the SDG40 gene is selected from the group consisting of: SDG40 gene of rice (XP_015644803.1), SDG40 gene of wheat (EMS51054.1), Arabidopsis (AT5G17240), tobacco (XM_016608916.1), SDG40 gene of corn (LOC100279317) and a combination thereof.

In another preferred embodiment, the amino acid sequence of the SDG40 protein is selected from the group consisting of:

(i) a polypeptide having the amino acid sequence as shown in any one of SEQ ID NO.: 1, 31-33;

(ii) a polypeptide having the function of regulating an agronomic trait and derived from (i), and formed by substitution, deletion, or addition of one or more (for example 1-10) amino acid residue(s) with the amino acid sequence as shown in any one of SEQ ID NO: 1, 31-33; or (iii) a polypeptide in which the homology between the amino acid sequence and the amino acid sequence as shown in any one of SEQ ID NO: 1, 31-33 is ≥90% (preferably ≥95%, more preferably ≥98%) and having the function of regulating an agronomic trait.

In another preferred embodiment, the nucleotide sequence of the SDG40 gene is selected from the group consisting of:

(a) a polynucleotide encoding the polypeptide as shown in any one of SEQ ID NO.: 1, 31-33;

(b) a polynucleotide having a sequence as shown in any one of SEQ ID NO.: 2, 34-36;

(c) a polynucleotide having a nucleotide sequence of ≥95% (preferably ≥98%, more preferably ≥99%) homologous to the sequence as shown in any one of SEQ ID NO.: 2, 34-36;

(d) a polynucleotide truncating or adding 1-60 (preferably 1-30, more preferably 1-10) nucleotide(s) at the 5′end and/or 3′end of the polynucleotide as shown in any one of SEQ ID NO.: 2, 34-36;

(e) a polynucleotide complementary to the polynucleotide of any of (a) to (d).

In another preferred embodiment, the preparation or composition is further used to reduce the methylation level of Rubsico.

In another preferred embodiment, the preparation or composition is further used to improve the carboxylation efficiency of Rubsico.

In another preferred embodiment, the preparation or composition is further used to increase the growth rate and/or increase the leaf area index.

In a second aspect of the present invention, it provides a method for improving an agronomic trait of plant, comprising the steps:

reducing the expression level or activity of the SDG40 gene or the encoded protein thereof in the plant, thereby improving the agronomic trait of the plant.

In another preferred embodiment, the “improving the agronomic trait of the plant” comprises:

(i) improving low light utilization efficiency (A_{low}); and/or

(ii) increasing biomass; and/or

(iii) increasing the number of tillers; and/or

(iv) increasing the yield per plant; and/or

(v) increasing the plant height.

In another preferred embodiment, the “improving low light utilization efficiency (A_{low})” comprises the steps of mutating C in the promoter region of the SDG40 gene in the plant to T and/or mutating A to C, thereby increasing the low light utilization efficiency (A_{low}) in the plant.

In another preferred embodiment, the promoter region is Chr7: 16884900-16886900.

In another preferred embodiment, the sequence of the promoter region is as shown in SEQ ID NO.: 37.

In another preferred embodiment, mutating the C at positions 523 to 1751 (preferably positions 1723) in the promoter region of the SDG40 gene in the plant to T and/or mutating A at positions 1803 to 1914 (preferably positions 1845) to C, thereby increasing the low light utilization efficiency (A_{low}) in the plant.

In another preferred embodiment, the method is performed in low light.

In another preferred embodiment, the low light means that the light intensity is less than 500 μmolm⁻²s⁻¹; preferably, 50-500 μmolm⁻²s⁻¹; more preferably, 50-100 μmolm⁻²s⁻¹.

In another preferred embodiment, the method comprises administering an inhibitor of a SDG40 gene or an encoded polypeptide thereof to a plant.

In another preferred embodiment, the method includes the steps:

(i) providing a plant or plant cell; and

(ii) introducing an inhibitor of a SDG40 gene or an encoded polypeptide thereof into the plant or plant cell, thereby obtaining a transgenic plant or plant cell.

In another preferred embodiment, the inhibitor is selected from the group consisting of: antisense nucleic acid, antibody, small molecule compound, Crispr reagent, siRNA, shRNA, miRNA, small molecule ligand, and a combination thereof.

In a third aspect of the present invention, it provides a method for improving the low light utilization efficiency (A_{low}) of a plant, comprising the steps of: reducing the expression of a SDG40 gene or an encoded protein thereof in the cell or plant, or mutating the C in the promoter region of the SDG40 gene in the plant to T and/or mutating A to C, thereby improving the low light utilization efficiency (A_{low}) of the plant.

In another preferred embodiment, the sequence of the promoter region is as shown in SEQ ID NO.: 37.

In another preferred embodiment, mutating the C at positions 523 to 1751 (preferably positions 1723) in the promoter region of the SDG40 gene in the plant to T and/or mutating A at positions 1803 to 1914 (preferably positions 1845) to C, thereby increasing the low light utilization efficiency (A_{low}) in the plant.

In a fourth aspect of the present invention, it provides a transgenic plant into which an inhibitor of a SDG40 gene or an encoded polypeptide thereof is introduced.

In another preferred embodiment, the inhibitor is selected from the group consisting of: antisense nucleic acid, antibody, small molecule compound, Crispr reagent, siRNA, shRNA, miRNA, small molecule ligand, and a combination thereof.

It should be understood that, within the scope of the present invention, each technical feature of the present invention described above and in the following (as examples) may be combined with each other to form a new or preferred technical solution, which is not listed here due to space limitations.

### DESCRIPTION OF FIGURE

FIG. 1 shows a result of the genome-wide association analysis of the low photosynthetic efficiency phenotype (A_{low}), as well as the natural variation (A) and population distribution (B) of A_{low}, and the Manhattan plot (C) and QQ plot (D) of A_{low} and a list of candidate genes within 50 KB upstream and downstream at the highest SNP peak (7m16911835) (E).

FIG. 2 shows a gene structure and haplotype analysis result of SDG40. Among them, a total of 2 significant SNPs are identified in the gene promoter region of SDG40 (A); there are 2 haplotypes in total, and there are 104 individuals in the haplotype of TC, and its A_{low} is significantly higher than 102 individuals of CA.

FIG. 3 shows the relationship between SDG40 gene down-regulation and A_{low} and other morphological traits, as well as the A_{low} phenotype distribution of the amiRNA-sdg transgenic T1 generation (A) and the correlation between the expression level of sdg gene and different transgenic lines (B); Analysis of differences (C) and image difference (D) in A_{low}, biomass, tiller number and yield per plant between the T3 generation homozygous line amiRNA2-1-3 of amiRNA-sdg and the wild type. Among them, 1-3, 1-5, 2-1 are the three lines that are transgenic positive for hygromycin identification, the mock is the negative line, and WT is the wild type.

FIG. 4 shows the essential information of the CRISPR homozygous mutant of SDG. Mutation location and sequencing information (A), SDG gene length and guide RNA recognition location (B).

FIG. 5 shows the relationship between the methylation of gene down-regulated and knocked-out transgenic lines and the maximum carboxylation efficiency of Rubisco, as well as the difference in the expression level of SDG40 gene in different transgenic lines (A) and the difference in the methylation level of Rubisco (C), changes of photosynthesis-intercellular CO₂ response curve (B) and the difference in the theoretical Rubisco maximum carboxylation efficiency (D).

FIG. 6 shows the phenotypic difference of Crispr-sdg grown under low light, and the picture between the wild-type rice and the gene knockout line grown in low light during the filling stage (A) and differences in specific photosynthetic and morphological parameters (B).

FIG. 7 shows the growth performance of SDG40 Arabidopsis mutants under low light. A: The performance of Arabidopsis wild type (col) and mutant (Atsdg40) grown in low light (LL, 100 μmol m⁻²s⁻¹) and high light (HL, 500 μmol m⁻²s⁻¹). B: Comparison of photosynthetic rate and biomass between wild-type and SDG homologous gene AT5G17240 Arabidopsis mutant (stock #: SALK_097673.56.00.X); C: Comparison of Rubisco methylation levels between wild-type and mutants by western blot. A pan-methylated antibody (PTM-602, PTM-Biolab, Hangzhou Jingjie Corp.) is used for the test (dilution ratio of 1:10000). CBB: Commassie blue staining.

FIG. 8 shows that the lack of SDG gene function increases the low light photosynthetic efficiency of corn. A: a primer sequence for editing a sequence of SDG homologous gene (LOC100279317) in maize through CRISPR-CAS9 technology; B: Sequence comparison analysis of B73 and 2 CRISPR knockout lines; C: Protein sequence comparison of rice ChSDG protein and corn ZmSDG. The editing position of CRISPR-CAS9 is marked with a box; D: Comparison of photosynthetic parameters and morphological characteristics of B73 and SDG maize mutants. Asat (photosynthetic efficiency under saturated light 1800PPFD), Alow (photosynthetic efficiency under low light 100PPFD), plant height (60-day plant height); E: Field performance of B73 and SDG maize mutants. The photo is taken at Lingshui base in Hainan, 60 days after sowing.

FIG. 9 shows that the lack of SDG gene function increases low light photosynthetic efficiency of tobacco. A: Phenotype comparison of CRISPR knockout lines (ntsdg) of Nicotiana benthamiana and NtSDG gene LOC107787360 in different periods; B: Sequence alignment information of ntsdg mutants and Nicotiana benthamiana; C: primer sequences identified by CRISPR knockout lines; D-E: Sequence similarity score and sequence analysis of rice ChSDG protein and

NtSDG protein, CRISPR-CAS9 editing position is marked with a box; F: Comparison of photosynthetic efficiency (Alow) of Nicotiana benthamiana (WT) and ntsdg under 1000PPFD saturated light (Asat) and 100PPFD low light. Different letters indicate significant differences in t-test (p<0.05).

FIG. 10 shows the sequence alignment analysis of SETdomain and rubisco binding domain in different species.

### DETAILED DESCRIPTION

Upon extensive and intensive studies and experiments, the inventors have discovered a SDG40 gene or an encoded protein thereof for the first time through research and screening of a large number of plant agronomic trait sites. The protein encoded by it is a methyltransferase. When inhibiting the expression of SDG40 gene or its encoded protein, it can significantly improve the agronomic traits of plants, including: (i) improving low light utilization efficiency (A_{low}); (ii) increasing biomass; (iii) increasing the number of tillers; (iv) increasing the yield per plant; (v) increasing the plant height. In addition, further experiments have also found that the mutation of C at positions 523 to 1751 (preferably positions 1723) in the promoter region of the SDG40 gene to T and/or mutation of A at positions 1803 to 1914 (preferably positions 1845) to C can also significantly improving low light utilization efficiency (A_{low}) in plant. On this basis, the inventors have completed the present invention.

SDG40 Gene

As used herein, the terms “SDG40 gene of the present invention” and “SDG40 gene” can be used interchangeably, and both refer to a SDG40 gene or a variant thereof derived from crops (such as rice and wheat). In a preferred embodiment, the nucleotide sequence of the SDG40 gene of the present invention is shown in any one of SEQ ID NO.: 2, 34-36. In the present invention, SEQ ID NO.: 37 is the sequence of the promoter region of SDG40 gene.

The present invention also includes a nucleic acid having 50% or more (preferably 60% or more, 70% or more, 80% or more, more preferably 90% or more, more preferably 95% or more, most preferably 98% or more, such as 99%) homology with the preferred gene sequence of the present invention (SEQ ID NO.: 2, 34-36), which can also effectively regulate the agronomic traits of crops (such as rice). “Homology” refers to the level of similarity (i.e., sequence similarity or identity) between two or more nucleic acids according to the percentage of the same position. Herein, variants of the gene can be obtained by inserting or deleting regulatory regions, performing random or site-directed mutations, and the like.

In the present invention, the nucleotide sequence in SEQ ID NO.: 2, 34-36 can be substituted, deleted or added one or more to generate the derivative sequence of SEQ ID NO.: 2, 34-36. Due to the degeneracy of the codons, even if the homology of which with SEQ ID NO.: 2, 34-36 is low, it can basically encode the amino acid sequence as shown in any one of SEQ ID NO.: 1, 31-33. In addition, the meaning of “the nucleotide sequence in SEQ ID NO.: 2, 34-36 has been substituted, deleted or added at least one nucleotide derivative sequence” also includes a nucleotide sequence that can hybridize to the nucleotide sequence as shown in SEQ ID NO.: 2, 34-36 under moderately stringent conditions; more preferably, under severely stringent conditions. These variant forms include (but are not limited to): the deletion, insertion and/or substitution of several (usually 1-90, preferably 1-60, more preferably 1-20, and most preferably 1-10) nucleotides, and the addition of several (usually within 60, preferably within 30, more preferably within 10, most preferably within 5) nucleotides at the 5′ and/or 3′ end.

It should be understood that although the genes provided in the examples of the present invention are derived from rice, however, the SDG40 gene sequence derived from other similar plants (especially plants belonging to the same family or genus as rice) and having certain homology (conservative) with the sequence of the present invention (preferably, the sequence is shown in SEQ ID NO.: 2, 34-36) is also included in the scope of the present invention, as long as those skilled in the art can easily isolate the sequence from other plants based on the information provided in this application after reading this application.

The polynucleotide of the present invention may be in the form of DNA or RNA. DNA forms include: DNA, genomic DNA, or synthetic DNA. DNA can be single-stranded or double-stranded. DNA can be a coding strand or a non-coding strand. The coding region sequence encoding the mature polypeptide may be the same as the coding region sequence shown in SEQ ID NO.: 2, 34-36 or a degenerate variant.

A polynucleotide encoding a mature polypeptide includes: a coding sequence that only encodes the mature polypeptide; the coding sequence of the mature polypeptide and various additional coding sequences; the coding sequence (and optional additional coding sequence) of the mature polypeptide and non-coding sequences.

The term “polynucleotide encoding a polypeptide” may include a polynucleotide encoding the polypeptide, or a polynucleotide that also includes additional coding and/or non-coding sequences. The present invention also relates to variants of the above polynucleotides, which encode fragments, analogs and derivatives of polyglycosides or polypeptides having the same amino acid sequence as the present invention. The variants of this polynucleotide can be naturally occurring allelic variants or non-naturally occurring variants. These nucleotide variants include substitution variants, deletion variants and insertion variants. As known in the art, an allelic variant is an alternative form of a polynucleotide, which may be a substitution, deletion or insertion of one or more nucleotides, but does not substantially change the function of the encoded polypeptide thereof.

The present invention also relates to polynucleotides that hybridize with the aforementioned sequences and have at least 50%, preferably at least 70%, and more preferably at least 80% identity between the two sequences. The present invention particularly relates to polynucleotides that can hybridize with the polynucleotide of the present invention under stringent conditions. In the present invention, “stringent conditions” refer to: (1) hybridization and elution at lower ionic intensity and higher temperature, such as 0.2×SSC, 0.1% SDS, 60° C.; or (2) adding denaturant during hybridization, such as 50% (v/v) methylphthalamide, 0.1% calf serum/0.1% Ficoll, 42° C., etc.; or (3) hybridization occurs only when the identity between the two sequences is at least 90% or more, and more preferably 95% or more.

It should be understood that although the SDG40 gene of the present invention is preferably derived from rice, other genes from other plants that are highly homologous to the rice SDG40 gene (such as having more than 80%, such as 85%, 90%, 95% or even 98% sequence identity) are also within the scope of the present invention. Methods and tools for aligning sequence identity are also well known in the art, such as BLAST.

The full-length sequence of SDG40 nucleotide of the present invention or its fragments can usually be obtained by PCR amplification method, recombinant method or artificial synthesis method. For the PCR amplification method, primers can be designed according to the relevant nucleotide sequence disclosed in the present invention, especially the open reading frame sequence, and a commercially available DNA library or a cDNA library prepared according to a conventional method known to those skilled in the art is used as a template to amplify the relevant sequence. When the sequence is long, it is often necessary to perform two or more PCR amplifications, and then splice the amplified fragments together in the correct order. Once the relevant sequence is obtained, the recombination method can be used to obtain the relevant sequence in large quantities. It is usually cloned into a vector, and then transferred into a cell, and then the relevant sequence is isolated from the proliferated host cell by conventional methods.

In addition, artificial synthesis methods can also be used to synthesize related sequences, especially when the fragment length is short. Usually, by first synthesizing multiple small fragments, and then ligating to obtain a very long fragment. At present, the DNA sequence encoding the protein (or fragment or derivative thereof) of the present invention can be obtained completely through chemical synthesis. This DNA sequence can then be introduced into various existing DNA molecules (or such as vectors) and cells known in the art. In addition, mutations can also be introduced into the protein sequence of the present invention through chemical synthesis.

Polypeptide Encoded by SDG40 Gene

As used herein, the terms “polypeptide of the present invention” and “coding protein of SDG40 gene” can be used interchangeably, and both refer to the polypeptide and its variants of SDG40 derived from rice. In a preferred embodiment, a typical amino acid sequence of the polypeptide of the present invention is shown in any one of SEQ ID NO.: 1, 31-33.

The present invention relates to an SDG40 polypeptide and variants thereof that regulate agronomic traits. In a preferred embodiment of the present invention, the amino acid sequence of the polypeptide is as shown in any one of SEQ ID NO.: 1, 31-33. The polypeptide of the present invention can effectively regulate the agronomic traits of crops (such as rice).

The present invention also includes polypeptides or proteins with the same or similar functions that have 50% or more (preferably 60% or more, 70% or more, 80% or more, more preferably 90% or more, more preferably 90% or more, more preferably 95% or more, most preferably 98% or more, such as 99%) homology with the sequence as shown in any one of SEQ ID NO.: 1, 31-33 of the present invention.

The “same or similar function” mainly refers to: “regulation of the agronomic traits of crops (such as rice)”.

The polypeptide of the present invention can be a recombinant polypeptide, a natural polypeptide, or a synthetic polypeptide. The polypeptide of the present invention can be a natural purified product, or a chemically synthesized product, or produced from a prokaryotic or eukaryotic host (for example, bacteria, yeast, higher plants, insect and mammalian cells) using recombinant technology. Depending on the host used in the recombinant production protocol, the polypeptide of the present invention may be glycosylated or non-glycosylated. The polypeptide of the present invention may also include or exclude the initial methionine residue.

The present invention also includes SDG40 protein fragments and analogs having SDG40 protein activity. As used herein, the terms “fragment” and “analog” refer to polypeptides that substantially maintain the same biological function or activity as the native SDG40 protein of the present invention.

The polypeptide fragments, derivatives or analogs of the present invention may be: (i) a polypeptide in which one or more conservative or non-conservative amino acid residues (preferably conservative amino acid residues) have been substituted, and such substituted amino acid residues may or may not be encoded by the genetic code; or (ii) a polypeptide with substitution groups in one or more amino acid residues; or (iii) a polypeptide formed by fusing a mature polypeptide with another compound (such as a compound that extends the half-life of the polypeptide, such as polyethylene glycol); or (iv) a polypeptide formed by fusing an additional amino acid sequence to this polypeptide sequence (such as a leader sequence or secretory sequence or a sequence or proprotein sequence used to purify the polypeptide, or fusion protein). According to the definition herein, these fragments, derivatives and analogs belong to the scope well known to those skilled in the art.

In the present invention, the polypeptide variant is the amino acid sequence as shown in any one of SEQ ID NO.: 1, 31-33, a derivative sequence obtained by replacing, deleting or adding at least one amino acid by several (usually 1-60, preferably 1-30, more preferably 1-20, most preferably 1-10), and adding one or several (usually within 20, preferably within 10, more preferably within 5) amino acids at the C-terminal and/or N-terminal. For example, in the protein, when amino acids with similar or close properties are substituted, the function of the protein is usually not changed, and the addition of one or several amino acids to the C-terminal and/or \terminal usually does not change the function of the protein. These conservative variants are best generated by substitution according to Table 1.

TABLE 1

| Initial | Representative | Preferred |
|---|---|---|
| residue | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also includes analogs of the claimed protein. The difference between these analogs and any one of the natural SEQ ID NO.: 1, 31-33 may be a difference in the amino acid sequence, or a difference in the modified form that does not affect the sequence, or both. Analogues of these proteins include natural or induced genetic variants. Induced variants can be obtained through various techniques, such as random mutagenesis through radiation or exposure to mutagens, site-directed mutagenesis or other known molecular biology technology. Analogues also include analogues having residues different from natural L-amino acids (such as D-amino acids), and analogues having non-naturally occurring or synthetic amino acids (such as β, γ-amino acids). It should be understood that the protein of the present invention is not limited to the representative proteins exemplified above.

Modified (usually not changing the primary structure) forms include: chemically derived forms of proteins in vivo or in vitro, such as acetoxylation or carboxylation. Modifications also include glycosylation, such as those that undergo glycosylation modifications during protein synthesis and processing. This modification can be accomplished by exposing the protein to an enzyme that performs glycosylation (such as a mammalian glycosylase or deglycosylase). Modified forms also include sequences with phosphorylated amino acid residues (such as phosphotyrosine, phosphoserine, phosphothreonine).

In addition, in the present invention, it can be seen from FIG. 10 that SET domain and rubisco binding domain have conserved functional regions in the species of the present invention (such as gramineous plants, cruciferous plants, Hibiscus mutabilis, leguminous plants, Solanaceae plants, cucurbitaceous plants, rosaceous plants, Chenopodiaceae plants, compositae plants, salicaceae plants, myrtaceae plants, papilionaceae plants, etc.). It can be speculated that the SDG proteins of these species can modify rubisco methylation similar to rice.

Expression Vector

The present invention also relates to a vector containing the polynucleotide of the present invention, a host cell produced by genetic engineering using the vector of the present invention or the mutant protein coding sequence of the present invention, and a method for producing the polypeptide of the present invention through recombinant technology.

Through conventional recombinant DNA technology, the polynucleotide sequence of the present invention can be used to express or produce recombinant mutant protein. Generally speaking, there are the following steps:

(1) Using the polynucleotide (or variant) of the present invention encoding the mutant protein of the present invention, or using a recombinant expression vector containing the polynucleotide to transform or transduce a suitable host cell;

(2). Culturing the host cell in a suitable medium;

(3). Separating and purifying the protein from culture medium or cells.

The present invention also provides a recombinant vector including the gene of the invention. As a preferred way, the downstream of the promoter of the recombinant vector contains a multiple cloning site or at least one restriction enzyme cutting site. When it is necessary to express the target gene of the present invention, the target gene is ligated into a suitable multiple cloning site or restriction enzyme cutting site, so that the target gene and the promoter are operably linked. As another preferred way, the recombinant vector includes (from 5′ to 3′ direction): a promoter, a target gene, and a terminator. If necessary, the recombinant vector may also include elements selected from the group consisting of: 3′ polynucleotide signal; untranslated nucleic acid sequence; transport and targeting nucleic acid sequence; resistance selection marker (dihydrofolate reductase, Neomycin resistance, hygromycin resistance and green fluorescent protein, etc.); enhancer; or operator.

In the present invention, the polynucleotide sequence encoding the mutant protein can be inserted into a recombinant expression vector. The term “recombinant expression vector” refers to bacterial plasmids, phages, yeast plasmids, plant cell viruses, mammalian cell viruses such as adenovirus, retrovirus, or other vectors well known in the art. As long as it can replicate and stabilize in the host, any plasmid and vector can be used. An important feature of an expression vector is that it usually contains an origin of replication, a promoter, a marker gene, and a translation control element.

Methods well known to those skilled in the art can be used to construct an expression vector containing the DNA sequence encoding the mutant protein of the present invention and appropriate transcription/translation control signals. These methods include in vitro recombinant DNA technology, DNA synthesis technology, and in vivo recombination technology. The DNA sequence can be effectively linked to an appropriate promoter in the expression vector to guide mRNA synthesis. Representative examples of these promoters are: Escherichia coli lac or trp promoter; lambda phage PL promoter; eukaryotic promoters including CMV immediate early promoter, HSV thymidine kinase promoter, early and late SV40 promoter, LTRs of retroviruses and some other known promoters that can control gene expression in prokaryotic or eukaryotic cells or viruses. The expression vector also includes a ribosome binding site for translation initiation and a transcription terminator.

Those of ordinary skill in the art can use well-known methods to construct expression vectors containing the genes of the present invention. These methods include in vitro recombinant DNA technology, DNA synthesis technology, and in vivo recombination technology. When constructing a recombinant expression vector using the gene of the present invention, any enhanced, constitutive, tissue-specific or inducible promoter can be added before the transcription initiation nucleotide.

The vector including the gene, expression cassette or of the present invention can be used to transform a suitable host cell so that the host expresses the protein. The host cell can be a prokaryotic cell, such as Escherichia coli, Streptomyces, Agrobacterium; or a lower eukaryotic cell, such as a yeast cell; or a higher eukaryotic cell, such as a plant cell. Those of ordinary skill in the art know how to select appropriate vectors and host cells. Transformation of host cells with recombinant DNA can be performed by conventional techniques well known to those skilled in the art. When the host is a prokaryote (such as Escherichia coli), it can be treated with CaCl₂ method or electroporation method. When the host is a eukaryote, the following DNA transfection methods can be selected: calcium phosphate co-precipitation method, conventional mechanical methods (such as microinjection, electroporation, liposome packaging, etc.). Agrobacterium transformation or gene gun transformation can also be used to transform plants, such as leaf disc method, immature embryo transformation method, flower bud soaking method, etc. The transformed plant cells, tissues or organs can be regenerated by conventional methods to obtain transgenic plants.

In addition, the expression vector preferably contains one or more selectable marker genes to provide phenotypic traits for selecting transformed host cells, such as dihydrofolate reductase for eukaryotic cell culture, neomycin resistance, and green fluorescent protein (GFP), or tetracycline or ampicillin resistance for E. coli.

A vector containing the above-mentioned appropriate DNA sequence and an appropriate promoter or control sequence can be used to transform an appropriate host cell so that it can express the protein.

The host cell can be a prokaryotic cell, such as a bacterial cell; or a lower eukaryotic cell, such as a yeast cell; or a higher eukaryotic cell, such as a mammalian cell. Representative examples include: Escherichia coli, Streptomyces; bacterial cells of Salmonella typhimurium; fungal cells such as yeast and plant cells (such as rice cells).

When the polynucleotide of the present invention is expressed in higher eukaryotic cells, if an enhancer sequence is inserted into the vector, the transcription will be enhanced. Enhancers are cis-acting factors of DNA, usually about 10 to 300 base pairs, acting on promoters to enhance gene transcription. Examples include SV40 enhancers of 100 to 270 base pairs on the late side of the replication initiation point, polyoma enhancers on the late side of the replication initiation point, and adenovirus enhancers, etc.

Those of ordinary skill in the art know how to select appropriate vectors, promoters, enhancers and host cells.

Transformation of host cells with recombinant DNA can be performed by conventional techniques well known to those skilled in the art. When the host is a prokaryotic organism such as Escherichia coli, competent cells that can absorb DNA can be harvested after the exponential growth phase and treated with the CaCl₂ method. The steps used are well known in the art. Another method is to use MgCl₂. If necessary, transformation can also be performed by electroporation.

When the host is a eukaryote, the following DNA transfection methods can be selected: calcium phosphate co-precipitation method, conventional mechanical methods such as microinjection, electroporation, liposome packaging, etc.

The obtained transformants can be cultured by conventional methods to express the polypeptide encoded by the gene of the present invention. Depending on the host cell used, the medium used in the culture can be selected from various conventional mediums. The culture is carried out under conditions suitable for the growth of the host cell. After the host cell has grown to an appropriate cell density, the selected promoter is induced by a suitable method (such as temperature conversion or chemical induction), and the cell is recultured for a period of time.

The recombinant polypeptide in the above method can be expressed in the cell or on the cell membrane, or secreted out of the cell. If necessary, the recombinant protein can be separated and purified by various separation methods using its physical, chemical and other characteristics. These methods are well known to those skilled in the art. Examples of these methods include, but are not limited to: conventional renaturation treatment, treatment with protein precipitation agent (salting out method), centrifugation, breaking bacteria through osmosis, ultra-treatment, ultra-centrifugation, molecular sieve chromatography (gel filtration), adsorption chromatography, ion exchange chromatography, high performance liquid chromatography (HPLC) and various other liquid chromatography techniques and the combination of these methods.

The main advantages of the present invention include:

(1) The present invention screens for the first time a SETDOMAIN40 (SDG40) gene, which encodes a chloroplast protein methyltransferase (OsCPMT1), which can regulate the activity of RUBISCO and other photosynthetic carbon metabolism enzymes.

(2) The present invention has found for the first time that reducing the expression of SDG40 gene or the encoded protein (especially under low light) can significantly improve the agronomic traits of plants, such as improving low light utilization efficiency (A_{low}), increasing the biomass, increasing the number of tillers, increasing the yield per plant, increasing the plant height, etc.

(3) The present invention has discovered for the first time that the mutation of C from position 523 to 1751 (preferably position 1723) in the promoter region of SDG40 gene to T and/or the mutation of A from position 1803-1914 (preferably position 1845) to C can significantly improve low light utilization efficiency (A_{low}) of plants.

(4) The present invention has found for the first time that reducing the expression of the SDG40 gene or the encoded protein thereof can significantly reduce the methylation level of Rubsico and improve the carboxylation efficiency of Rubisco.

(5) The present invention has found for the first time that reducing the expression of the SDG40 gene or the encoded protein thereof can also increase the growth rate and/or increase the leaf area index.

The present invention is further described below with reference to specific embodiments. It should be understood that these examples are only for illustrating the present invention and not intended to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions described in the Journal of Microbiology: An Experimental Handbook (edited by James Cappuccino and Natalie Sherman, Pearson Education Press) or the manufacturer's proposed conditions. Unless otherwise stated, the materials and reagents used in the examples are all commercially available products.

General Method

1. Measurement of Low Light Utilization Efficiency A_{low}

In the genome-wide association analysis, the small core natural population of minicore rice is used as the material. This population contains 205 rice lines or varieties (purchased from the USDA Germplasm Resource Bank, USDA-Genetic Stocks Oryza) and comes from 97 countries around the world. The experiment was carried out in rice breeding at the Institute of Genetics and Development of the Chinese Academy of Sciences. The seeds were sown in mid-May 2013. The population was grown in potted conditions under natural light and watered twice a week. The photosynthesis measurement was started 60 days after sowing. In order to eliminate the influence of daytime temperature on the photosynthesis measurement, the material was moved into the artificial climate chamber in advance before the measurement, the room temperature was controlled at 27° C., and the light intensity was maintained at about 600PPFD. During the measurement, 4 portable photosynthesis instruments (LICOR-6400XT) were used simultaneously.

The leaf chamber temperature was 25° C., the light intensity was 100PPFD, and the CO₂ was 400 ppm. Each line was 4 biological replicates. The photosynthetic rate-intercellular CO₂ reaction curve determination was completed by an automatic program. Each curve was consisted of 14 CO₂ concentration gradient data points, first of which were 425, 350, 250, 150, 100, 40, 425, 500, 600, 700, 900, 1100, 1400, and 1800 ppm in order. The time interval for each data point was 5 minutes. The maximum carboxylation efficiency (V_{cmax}) of Rubisco was estimated based on the Farquhar photosynthetic biochemical model (Farquhar et al. 1980).

2. Genome-Wide Association Analysis and Candidate Gene Screening

After quality control and SNP filtering, a total of 2.3M SNPs were obtained for genome-wide association analysis (GWAS). GWAS is implemented by the conventional GEMAA software, and a hybrid linear model algorithm is used for correlation analysis. After 200 random samples, the significance threshold of association analysis is defined (P value=6), and then GCTA open source software (Jian Yang University of Queensland university, http://cnsgenomics.com/software/gcta/index.html) was used to calculate the linkage disequilibrium distance of the highest SNP peak (7m16911835). Both Manhattan and QQ maps are completed by the open source software R (R 3.2.1 GUI 1.66 Mavericks build).

In order to dig deeper into the candidate genes, 10 lines each with the extreme phenotype A_{low} were selected, and 12 candidate genes near the highest SNP were determined (Table 1).

TABLE 1
Difference analysis of the expression levels of candidate genes in different extreme materials

| qPCR against actin | | hsp | ep-1 | ribo | ep-2 | sdg | ep-3 | ep-4 | ep-5 | erf | ppr | zinc | ep-6 | Alow |
|---|---|---|---|---|---|---|---|---|---|---|---|---|---|---|
| AUS | S4161 | 2.3 | 0.0 | 13.4 | 0.2 | 4.6 | 0.0 | 0.0 | 0.0 | 12.4 | 2.1 | 3.4 | 0.0 | 5.7 |
| TEJ | G4064 | 4.5 | 0.0 | 0.4 | 0.0 | 5.7 | 0.0 | 0.0 | 0.0 | 3.5 | 0.4 | 13.4 | 0.0 | 5.8 |
| AUS | T4178 | 2.4 | 0.2 | 11.3 | 0.0 | 18.5 | 0.0 | 0.0 | 1.3 | 11.0 | 1.0 | 0.5 | 0.0 | 5.8 |
| TEJ | L4101 | 3.7 | 0.0 | 5.8 | 3.2 | 3.5 | 1.0 | 0.0 | 0.0 | 0.4 | 1.3 | 3.4 | 3.4 | 5.9 |
| AUS | P4133 | 5.8 | 0.1 | 3.5 | 0.0 | 2.6 | 0.2 | 0.0 | 0.0 | 3.4 | 3.4 | 2.5 | 0.0 | 5.9 |
| AUS | R4153 | 8.8 | 0.0 | 9.5 | 0.0 | 14.5 | 0.0 | 4.0 | 2.1 | 2.6 | 1.2 | 21.4 | 0.0 | 6.1 |
| AUS | Q4150 | 10.7 | 0.0 | 11.4 | 0.0 | 6.7 | 0.0 | 0.0 | 0.0 | 0.7 | 1.4 | 3.5 | 0.0 | 6.1 |
| AUS | R4152 | 10.4 | 0.0 | 13.4 | 1.2 | 3.5 | 0.0 | 0.0 | 0.0 | 3.6 | 0.5 | 3.4 | 2.4 | 6.2 |
| AUS | R4151 | 4.2 | 1.2 | 20.4 | 0.0 | 6.7 | 0.1 | 0.1 | 0.0 | 3.9 | 2.3 | 13.4 | 0.3 | 6.3 |
| Admix | P4134 | 7.8 | 0.1 | 22.1 | 0.0 | 4.5 | 0.0 | 0.0 | 0.4 | 2.4 | 1.4 | 0.4 | 0.0 | 6.4 |
| TRJ | N4130 | 8.5 | 0.0 | 0.4 | 0.0 | 15.4 | 0.0 | 0.0 | 0.0 | 10.3 | 3.4 | 11.4 | 9.0 | 2.6 |
| IND | X4203 | 5.2 | 0.0 | 4.6 | 0.0 | 12.5 | 0.0 | 0.0 | 0.0 | 14.5 | 0.5 | 3.5 | 0.0 | 2.6 |
| Admix | P4132 | 6.3 | 0.0 | 13.5 | 1.0 | 8.3 | 0.0 | 0.0 | 0.0 | 4.5 | 11.0 | 11.4 | 0.0 | 3.2 |
| AUS | N4129 | 15.6 | 0.0 | 22.5 | 1.3 | 17.8 | 0.5 | 0.0 | 2.1 | 3.5 | 0.4 | 0.5 | 2.4 | 3.2 |
| IND | E4044 | 9.1 | 0.1 | 0.5 | 0.0 | 9.6 | 0.0 | 0.0 | 0.0 | 8.9 | 3.4 | 3.4 | 0.0 | 3.3 |
| AUS | L4109 | 2.5 | 0.0 | 24.5 | 0.0 | 12.3 | 0.0 | 0.0 | 0.0 | 13.5 | 2.6 | 2.5 | 0.0 | 3.3 |
| IND | X4204 | 8.6 | 0.0 | 2.5 | 0.0 | 7.9 | 0.0 | 0.0 | 0.4 | 10.7 | 0.7 | 4.5 | 3.4 | 3.4 |
| AUS | T4171 | 13.5 | 0.2 | 23.5 | 0.0 | 9.6 | 0.0 | 0.4 | 0.0 | 4.5 | 3.6 | 3.3 | 2.3 | 3.5 |
| AUS | L4106 | 4.9 | 0.2 | 1.5 | 1.0 | 12.3 | 0.0 | 0.5 | 0.0 | 3.3 | 3.9 | 2.5 | 0.0 | 3.8 |
| IND | H4079 | 21.0 | 0.0 | 3.5 | 0.0 | 9.5 | 0.6 | 1.4 | 0.4 | 2.5 | 2.4 | 3.4 | 0.0 | 3.8 |
| High | | 6.06 | 0.16 | 11.11 | 0.46 | 7.08 | 0.13 | 0.41 | 0.38 | 4.39 | 1.50 | 6.53 | 0.61 | 6.02 |
| Low | | 9.52 | 0.05 | 9.69 | 0.33 | 11.52 | 0.11 | 0.23 | 0.29 | 7.62 | 3.19 | 4.64 | 1.71 | 3.27 |
| t-test | | 0.05 | 0.18 | 0.36 | 0.36 | 0.02 | 0.44 | 0.34 | 0.39 | 0.05 | 0.06 | 0.23 | 0.14 | 2.3E−13 |

The rice leaves 5 weeks after emergence were selected, and the samples were stored in liquid nitrogen. TRIzol Plus RNA Purification Kit (Yingwei Jieji Life Technology Company) was used for RNA extraction, according to the standard procedure of the manual. SuperScript VILO cDNA Reverse Transcription Kit (Yingwei Jieji Life Technology Company) was used for reverse transcription of cDNA. 2 ug of total RNA was used for reverse transcription of cDNA. Quantitative PCR was achieved using SYBR Green PCR reaction system (Applied Biosystems) and ABI quantitative PCR instrument (StepOnePlus). The amplification reaction program is: 95° C. 10 s, 55° C. 20 s, 72° C. 20 s. The housekeeping gene is actin. Three biological replicates and three technical replicates. The newly developed primer sequences are as follows (Table 2):

TABLE 2
Primer sequence list of quantitative PCR

| | | | | Product |
|---|---|---|---|---|
| | | | Tm | size |
| Gene | Primer | Primer sequence | (° C.) | (bp) |
| Ep-3 | F | CCTCGACGGCGATGTGG (SEQ ID NO.: 4) | 60.3 | 208 |
| | R | AAGGGGTCTTGTCCTTGTCA (SEQ ID NO.: 5) | 58.5 | |
| EP-4 | F | CCACGGGTTCACCAACTTGA (SEQ ID NO.: 6) | 60.5 | 202 |
| | R | CCAAAGTTCGACTTGGAATGACA (SEQ ID NO.: 7) | 59.4 | |
| PPR | F | GTTTCCATGAGCACCTTCGT (SEQ ID NO.: 8) | 60.1 | 162 |
| | R | CTCAAGCAAGAACTGCATCG (SEQ ID NO.: 9) | 59.7 | |
| ZINC | F | TGCCGTAACCTGCTCATGTA (SEQ ID NO.: 10) | 60.3 | 157 |
| | R | GAGCCCTGAAGCCATTTGTA (SEQ ID NO.: 11) | 60.2 | |
| EP-6 | F | TTGACTTTGGCAGCAGTGAC (SEQ ID NO.: 12) | 60.0 | 158 |
| | R | CATATTCAATGGCGCAGATG (SEQ ID NO.: 13) | 60.1 | |
| ERF | F | GGAGCACGAAGAAGTCCAAG (SEQ ID NO.: 14) | 60.0 | 250 |
| | R | CCTCCCCATGCATTGTAATC (SEQ ID NO.: 15) | 60.2 | |
| SDG | F | GCTCGTCCTTTTATGCAAGC (SEQ ID NO.: 16) | 60.0 | 169 |
| | R | CCATCTTTCCAGGGATCGTA (SEQ ID NO.: 17) | 59.9 | |
| EP2 | F | CGGTGTTCTAGCGAAACAGA (SEQ ID NO.: 18) | 59.1 | 194 |
| | R | CCGTATGTTCCATCATGTGC (SEQ ID NO.: 19) | 59.8 | |
| Ribo | F | CAATGCTGATAGCGGTGAGA (SEQ ID NO.: 20) | 60.0 | 241 |
| | R | GTGGCCATACCTCGCATAGT (SEQ ID NO.: 21) | 60.0 | |
| Ep-1 | F | GTGAGCGTCCCTCTCCTATG (SEQ ID NO.: 22) | 59.8 | 240 |
| | R | TCTCTTCCTCCTCAGGCTCA (SEQ ID NO.: 23) | 60.2 | |
| HSP | F | ATGAAGATGAACCGGAAACG (SEQ ID NO.: 24) | 59.9 | 187 |
| | R | GCCAAAGATACCTCCGTCTG (SEQ ID NO.: 25) | 59.7 | |
| actin | F | ACCATTGGTGCTGAGCGTTT (SEQ ID NO.: 26) | 58.9 | 268 |
| | R | CGCAGCTTCCATTCCTATGAA (SEQ ID NO.: 27) | 60.1 | |

3. Construction of CRISPR-CAS9 Vector System

The codon-optimized hSpCas9 and the corn ubiquitin promoter (UBI) were co-linked to the pCAMBIA1300 binary vector (purchased from NTCC Type Culture Collection-Biovector Plasmid Vector Strain Cell Protein Antibody Gene Collection). The vector backbone contains a hygromycin selection marker (HPT). The primer screening sequences are: F, AGCTGCGCCGATGGTTTCTACAA (SEQ ID NO.: 28); R, ATCGCCTCGCTCCAGTC AATG (SEQ ID NO.: 29). In order to construct a complete CRISPR/Cas9 binary vector pBGK032, additional OsU6 promoter, selection marker gene ccdB, restriction enzyme site with BsaI and sgRNA sequence derived from pX260 need to be introduced. The specific sequence that recognizes the CDS region of the sdg gene was completed by artificial synthesis. Finally, 10 ng of the digested pBGK032 vector and 0.05 mM oligo binder were connected, and 10 μl of the reaction system was used. After sequencing confirmed that there was no base mutation, then proceed to the next step, including E. coli expression plasmid, Agrobacterium tumefaciens-mediated rice transformation and callus regeneration system.

4. Construction of amiRNA Gene Interference System

Artificial microRNAs (amiRNAs) are 21mer small RNAs that can be used to specifically identify target genes to reduce gene expression levels. Based on the WMD3 MicroRNA design website (http://wmd3.weigelworld.org/) and the TIGR rice genome annotation website, we constructed a miR319 vector that specifically recognized the SDG40 gene. It consisted of three parts (5′ arm-centralloop-3′arm). First, the three fragments were amplified separately. Then, the 20mer sequence of miR319 was replaced by designing specific 21mer small RNAs (TCTTTGAGCAAGAATTTGCT SEQ ID NO.: 30). According to the WMD3 design, the pNW55 vector (purchased from the NTCC Type Culture Collection-Biovector Plasmid Vector Cell Protein Antibody Gene Collection) was used as a template for PCR amplification, then the gel was cut, purified and integrated into pGEMH-T Easy Vector (Promega). The restriction enzyme site was BamHI/KpnI. The obtained recombinant fragment was then ligated with the IRS154 binary vector (derived from pCAMBIA). After sequencing confirmed that there was no base mutation, the next step was carried out, including E. coli expression plasmid, Agrobacterium tumefaciens-mediated rice transformation and Callus regeneration system.

5. Agrobacterium-mediated Transgene and Mutant Detection

The constructed CRISPR/Cas9 and amiRNA plasmids were expressed in Agrobacterium tumefaciens strain EHA105 (purchased from NTCC Type Culture Collection-Biovector Plasmid Vector Cell Protein Antibody Gene Collection) by heat shock method. The choice of transformation recipient was generally callus induction from mature embryos of wild-type rice seed (Zhonghua 11) (purchased from Shanghai Guangming Seed Industry Co., Ltd.). After the induction medium was increased or decreased for 2 weeks, the embryos were cut off and cultured for 1 week, the vigorously growing callus was used as the recipient of transformation. Using conventional Agrobacterium-mediated genetic transformation methods, the EHA105 strain containing the above two plasmid vectors was infected with rice callus, and after co-cultivation in the dark at 25° C. for 3 days, they were cultured on a screening medium containing 120 mg/L G418. The selected resistant callus was cultured on a predifferentiation medium containing 120 mg/L for about 10 days. The pre-differentiated callus was transferred to differentiation medium and cultured under light conditions. Resistant transgenic plants were obtained in about a month.

6. Methylation Level Detection

The rice leaves 5 weeks after emergence were selected, and the samples were stored in liquid nitrogen. SDS protein extract contains: 25 mM Tris-HCl, pH 7.8, 1 mM EDTA, 5 mM MgCl₂, 1% (w/v) SDS, 2 mM β-mercaptoethanol). About 50 mg of fresh leaves were ground with liquid nitrogen and mixed with 1 ml of SDS protein extract, and was heated at 100° C. for 3-5 minutes. After centrifugation at 12,000 g for 10 minutes, the supernatant was extracted. 12% SDS-PAGE gel was used to separate about 5 μg protein. Coomassie brilliant blue staining was performed to observe changes in protein content. Nylon cellulose membrane was used as a medium for protein transfer in immunohybridization. It was blocked with 5% skimmed milk powder and then hybridized with a 1:5000 of pan-1,2-methylated antibody (ab23367, Abcam). Finally, the color was developed with chemiluminescent ECL, and the luminous photographic system of GE (LAS-4000 Mini, GE Healthcare) was used for filming

### EXAMPLE 1

### Large-Scale Low Light Utilization Efficiency Phenotype Survey and Genome-Wide Association Analysis (GWAS)

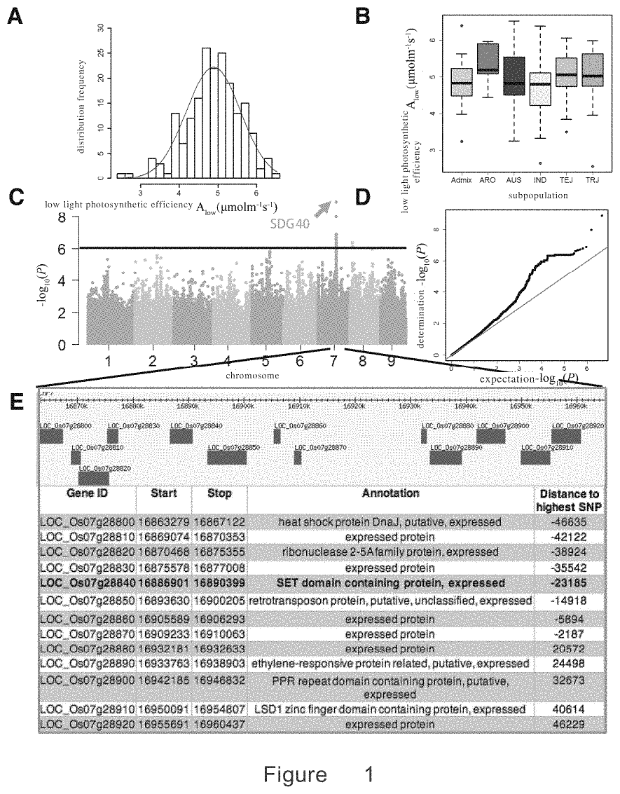

Using 217 rice natural minicore groups from 97 countries around the world, through years of multi-site experiments, the natural variation and subpopulation distribution of low light utilization efficiency (A_{low}) were investigated (FIG. 1A and FIG. 1B), and using the 2.3M filtered SNPs with whole genome coverage for association analysis to obtain A_{low}'s Manhattan and QQ maps (FIG. 1C&D). The highest SNP peak (7m16911835) is located on chromosome 7 with a P value of 2.3E-09. GCTA software was used to calculate the linkage disequilibrium distance (LD=50 KB) of the highest SNP peak. In the vicinity of 50 KB upstream and downstream of this peak, a total of 12 candidate genes have been found (FIG. 1E).

### EXAMPLE 2

### Preliminary Screening of Candidate Genes

10 high and low materials with extreme A_{low} phenotype were selected, and the expression differences of 12 candidate genes in individual materials with extreme phenotypes were analyzed by qPCR (Table 1). The results show that the SDG40 gene presents the most significant difference (pair-wise t-test, P value=0.02). Among them, in individual materials with low A_{low} phenotype, the average expression level of SDG40 gene is 64% higher than that of individual materials with high A_{low} phenotype, indicating that this gene may have a negative regulatory effect on low light utilization efficiency.

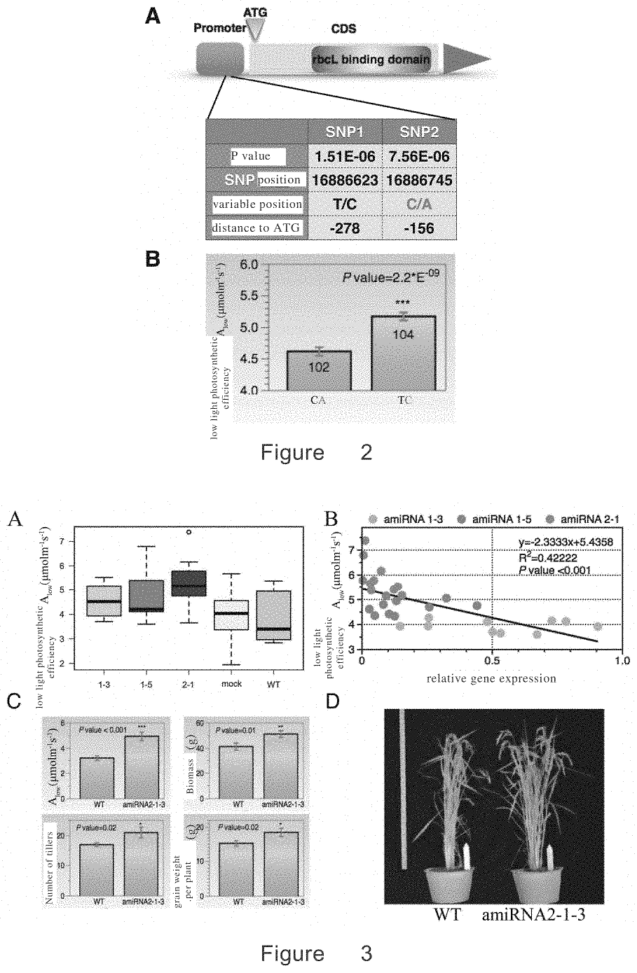

The present invention has also found that the difference in activity of the promoter region of SDG40 gene can lead to phenotypic differences. GWAS results show (FIG. 2, A-B) that there are two significant SNPs in the promoter region of SDG40 gene, 7m16886623 (T/C) and 7m16886745 (C/A), which correspond to positions 523 to 1751 (preferably positions 1723) and positions 1803 to 1914 (preferably positions 1845) in the promoter region (SEQ ID NO.: 3 and 37) of the SDG40 gene. Haplotype structure analysis has shown that the 104 subpopulations containing TC mutations and 102 subpopulations containing CA have significant changes in A_{low}. Among them, the A_{low} of 104 subpopulations containing TC mutation is significantly higher than that of 102 subpopulations containing CA, indicating that changes in expression activity caused by haplotype variation in the promoter region can cause changes in photosynthetic phenotype.

### EXAMPLE 3

### The Relationship Between SDG40 Gene Down-Regulation, Knock-Out and Photosynthetic Efficiency and Economic Yield

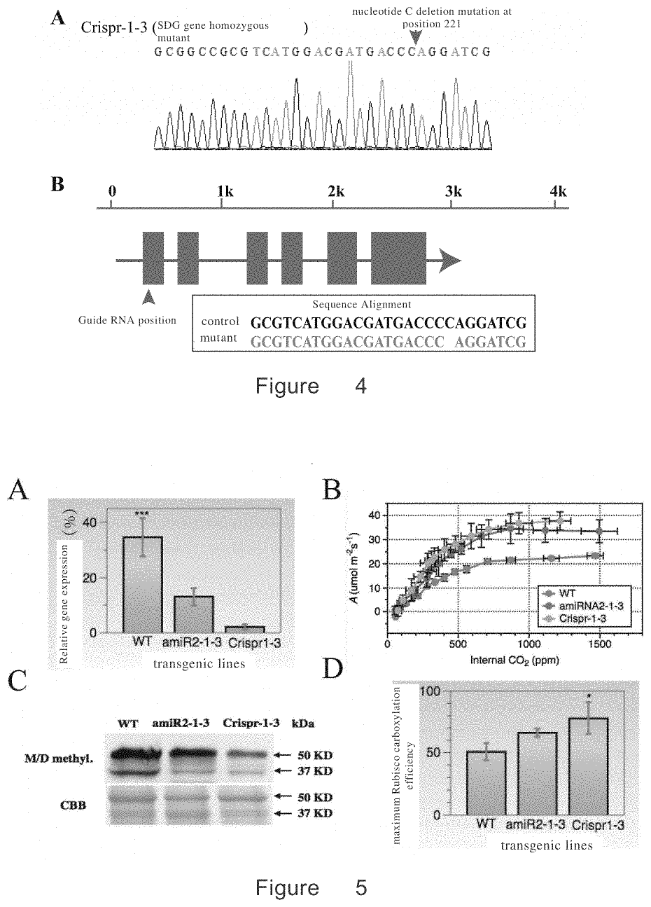

In order to prove the negative regulation relationship between SDG40 gene and rice leaf photosynthetic efficiency, CRISPR-CAS9 vector system and amiRNA gene interference vector system were used in combination with Agrobacterium transformation system to obtain transgenic pure line progeny materials. First, the comparison between the three different amiRNA lines of T1 generation and the wild-type A_{low} phenotype was determined (FIG. 3, A-D).

The results show that the low photosynthetic efficiency of the three amiRNA lines were significantly higher than the negative control (mock) and wild-type materials. With the increase of SDG40 gene expression level, the value of A_{low} shows a significant linear decreasing trend (R²=0.42). The phenotype of the homozygous line amiRNA2-1-3 of the T3 generation was also investigated, and it is found that the low light photosynthetic efficiency A_{low}, biomass, tiller number and yield per plant are all significantly higher than the control (FIG. 3C-D).

Since the protein encoded by SDG40 is a methyltransferase, in the present invention CRISPR gene editing technology was used to knock out the nucleotide sequence at position 221 of the SDG40 gene to obtain the homozygous mutant material of SDG40 (Crispr-1-3), and the changes in methylation levels were analyzed between transgenic lines with different gene expression levels (FIG. 4, A-B).

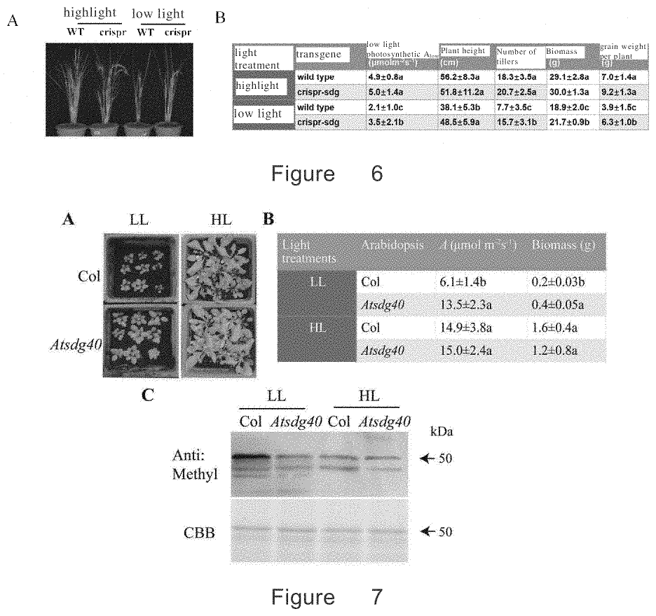

The results show that with the decrease of SDG40 gene expression, Rubisco's methylation level also decreases synchronously (FIG. 5A, C).

In order to analyze the relationship between the changes of Rubisco methylation level and carboxylation activity, the photosynthetic-intercellular CO₂ response curve between different transgenic lines was analyzed. The results show that the maximum carboxylation efficiency (Vcmax) of Rubisco shows a regular increasing trend with the decrease of SDG40 gene expression and Rubisco methylation level, indicating that the expression level of SDG40 gene can affect the methylation level of Rubisco, which in turn affects the carboxylation efficiency of Rubisco. (FIG. 5, A-D).

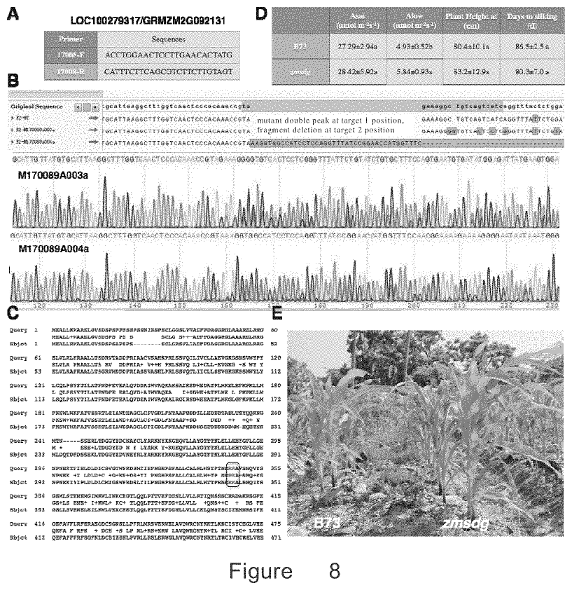

To further prove the low light advantage of SDG40 gene knockout transgenic lines, Crispr materials were grown under different light conditions (high light 1500PPFD and low light 100PPFD) (FIG. 6, A-B). The results show that Crispr materials exhibit a better growth condition under low light, including A_{low}, the plant height, tiller number, biomass and yield per plant, which are all significantly higher than the control. In bright light, the difference is not obvious (FIG. 6).

### EXAMPLE 4

### The Relationship Between the Down-Regulation, Knock-Out of SDG40 Gene in Arabidopsis and the Photosynthetic Efficiency and Economic Yield

Through the T-DNA insertion mutation technology, the 32nd amino acid mutation of AtSDG40 gene was caused.

The results are shown in FIG. 7. The results show that, compared with the wild-type Col, the mutant Atsdg40 of the AtSDG40 gene exhibits a better weak light advantage under low light, showing a higher photosynthetic efficiency, while in bright light, it is no different from the wild type (FIG. 7, A-B). Low-light treatment can reduce the biomass of the wild type by 33%, while for the mutant it only reduces the biomass by 12% (FIG. 7, B). The degree of Rubisco methylation of Arabidopsis wild-type under low light is significantly higher than that of Rubisco under high light. The Rubisco methylation level of the mutant is not significantly different under high light and low light (FIG. 7, C).

### EXAMPLE 5

### The Relationship Between Down-Regulation, Knock-Out of SDG40 Gene in Maize and Photosynthetic Efficiency and Economic Yield

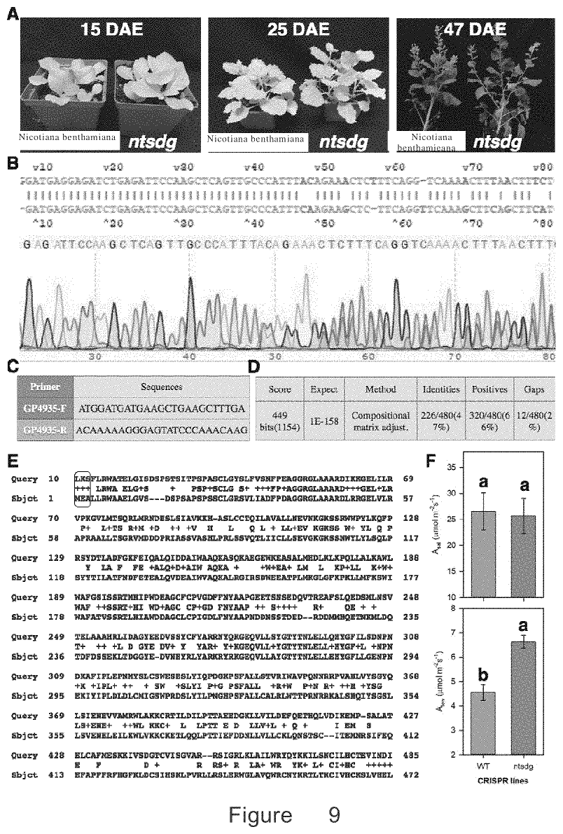

Using CRISPR-CAS9 technology, a site-directed mutation of the ZmSDG40 gene of B73 corn resulted in the loss of function of the gene. The gRNA sequence is: GCAAGTCACGCGCCGCCGCG. The results are shown in FIG. 8. The results show that through specific PCR amplification and sequencing, it is proved that the 349 amino acid insertion mutations of corn ZmSDG are successfully obtained by using CRISPR-CAS9 (FIG. 8, A-C). Multiplication is expanded to obtain T1 generation knockout lines. After single-strand knockout, the photosynthetic efficiency (Alow) under low light still increases to a certain extent by 12%, which reduces the flowering period of corn, but does not increase the photosynthetic efficiency and plant height under high light (FIG. 8, D-E).

### EXAMPLE 6

### The Relationship Between Down-Regulation, Knock-Out of SDG40 Gene in Tobacco and Photosynthetic Efficiency and Economic Yield

Using CRISPR-CAS9 technology, the SDG40 homologous gene in tobacco was knocked out, and the gene function was lost.

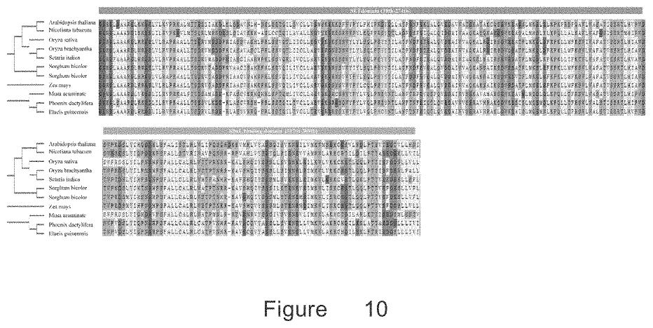

The results are shown in FIG. 9. The results show that the ninth amino acid of the tobacco SDG homologous gene LOC107787360 is knocked out using CRISPR-CAS9, named ntsdg (FIG. 9, B-E). This material has a faster growth rate and leaf area index (FIG. 9, A), higher low light photosynthetic efficiency, while the photosynthetic efficiency of ntsdg under saturated light does not increase significantly (FIG. 9, F).

All documents mentioned in the present invention are cited as references in this application, as if each document is individually cited as a reference. In addition, it should be understood that after reading the above teaching content of the present invention, those skilled in the art can make various changes or modifications to the present invention, and these equivalent forms also fall within the scope defined by the appended claims of the present application.

1. A method of regulating an agronomic trait of a plant, comprising the steps of administering an inhibitor of SDG40 gene or encoded protein thereof to a plant, wherein the agronomic trait of the plant is selected from one or more of the following groups:
(i) low light utilization efficiency (A_{low});
(ii) biomass;
(iii) the number of tillers;
(iv) yield per plant;
(v) plant height.

2. The method of claim 1, wherein “the regulation of an agronomic trait of a plant” comprises:
(i) the improvement of low light utilization efficiency (A_{low}); and/or
(ii) the increasement of biomass; and/or
(iii) the increasement of the number of tillers; and/or
(iv) the increasement of the yield per plant; and/or
(v) the increasement of plant height.

3. The method of claim 1, wherein the inhibitor is selected from the group consisting of: antisense nucleic acid, antibody, small molecule compound, Crispr reagent, siRNA, shRNA, miRNA, small molecule ligand, and a combination thereof.

4. The method of claim 1, wherein the SDG40 gene is from one or more crops from the following group: Gramineae, Solanaceae, and Cruciferae.

5. The method of claim 1, wherein the amino acid sequence of the SDG40 protein is selected from the group consisting of:
(i) a polypeptide having the amino acid sequence as shown in any one of SEQ ID NO.: 1, 31-33;
(ii) a polypeptide having the function of regulating an agronomic trait and derived from (i), and formed by substitution, deletion, or addition of one or more (for example 1-10) amino acid residue(s) with the amino acid sequence as shown in any one of SEQ ID NO: 1, 31-33; or (iii) a polypeptide in which the homology between the amino acid sequence and the amino acid sequence as shown in any one of SEQ ID NO: 1, 31-33 is ≥90% (preferably ≥95%, more preferably ≥98%) and having the function of regulating an agronomic trait.

6. The method of claim 1, wherein the nucleotide sequence of the SDG40 gene is selected from the group consisting of:
(a) a polynucleotide encoding the polypeptide as shown in any one of SEQ ID NO.: 1, 31-33;
(b) a polynucleotide having a sequence as shown in any one of SEQ ID NO.: 2, 34-36;
(c) a polynucleotide having a nucleotide sequence of ≥95% (preferably ≥98%, more preferably ≥99%) homologous to the sequence as shown in any one of SEQ ID NO.: 2, 34-36;
(d) a polynucleotide truncating or adding 1-60 (preferably 1-30, more preferably 1-10) nucleotide(s) at the 5′end and/or 3′end of the polynucleotide as shown in any one of SEQ ID NO.: 2, 34-36;
(e) a polynucleotide complementary to the polynucleotide of any of (a) to (d).

7. A method for improving an agronomic trait of plant, comprising the steps:
reducing the expression level or activity of the SDG40 gene or the encoded protein thereof in the plant, thereby improving the agronomic trait of the plant.

8. The method of claim 7, wherein the “improving the agronomic trait of the plant” comprises:
(i) improving low light utilization efficiency (A_{low}); and/or
(ii) increasing biomass; and/or
(iii) increasing the number of tillers; and/or
(iv) increasing the yield per plant; and/or
(v) increasing the plant height.

9. The method of claim 8, wherein the “improving low light utilization efficiency (A_{low})” comprises the step of mutating C in the promoter region of the SDG40 gene in the plant to T and/or mutating A to C, thereby increasing the low light utilization efficiency (A_{low}) in the plant.

10. A method for improving the low light utilization efficiency (A_{low}) of a plant, comprising the steps of: reducing the expression of a SDG40 gene or an encoded protein thereof in the cell or plant, or mutating the C in the promoter region of the SDG40 gene in the plant to T and/or mutating A to C, thereby improving the low light utilization efficiency (A_{low}) of the plant.


SEQUENCE LISTING (37 sequences)

SEQ ID NO: 1 - protein, 479 aa, Oryza sativa
MEALLRWAAELGVSDSPSAPSPSSCLGRSVLIADFPDAGGRGLAAARDLRRGELVLRAPRAALLTSGRVMDDDPRIASSVASHLPRLSSVQTLIICLLSEVGKGKSSNWYLYLSQLPSYYTILATFNDFETEALQVDEAIWVAQKALRGIRSDWEEATPLMKGLGFKPKLLMFKSWIWAFATVSSRTLHIAWDDAGCLCPIGDLFNYAAPNDDNSSTDEDRDDMMHQETNKMLDQTDFDSSEKLTDGGYEDVNEYRLYARKRYRKGEQVLLAYGTYTNLELLEHYGFLLGENPNEKIYIPLDLDLCMIGSWPRDSLYILPNGHPSFALLCALRLWTTPRNRRKALSHQIYSGSLLSVENELEILKWLVKKCKETLQQLPTTIEFDDNLLVLLCKLQNSTSCITEMNRSIFEQEFAPFFRFHGFKLDCSIHSKLPVRLLRSLERWGLAVQWRCNYKRTLTKCIVHCKSLVHELSLQQNQQ

SEQ ID NO: 2 - DNA, 1440 bp, Oryza sativa
atggaggccctcctccggtgggcggcggagctgggcgtctccgactccccgtccgcgccctccccctcctcctgcctcggccgctccgtcctcatcgccgacttccccgacgccggcgggaggggcttggcggcggcgcgggacctccggcgcggcgagctggtgctgcgggcgccgcgcgccgccctgctcaccagcggccgcgtcatggacgatgaccccaggatcgcctcctccgtcgccagccacctcccccgcctctcctccgtccagaccctgatcatatgtttgttgtctgaagtgggaaaagggaagagttcaaattggtatctatatttgtctcagctgccctcctactacactatcttggctaccttcaatgattttgaaactgaagctctccaagttgatgaggctatttgggttgctcaaaaggctcttcgtggaataagatcagactgggaagaagcaacaccactgatgaaagggttgggatttaaacctaagcttttgatgtttaaatcatggatctgggcttttgcaacggtatcttcaagaacattgcatatagcatgggatgatgctggttgtctatgtcctattggtgatttgttcaattatgctgctcctaatgatgacaattcatcaacggatgaagatagagatgatatgatgcatcaggagacaaataagatgttggaccagacagattttgattcatctgagaagttgacagatgggggatatgaagatgttaatgaataccgtctgtatgctcggaaaagatatagaaaaggagagcaggtacttctggcttatggaacgtatacaaacttggaacttcttgagcattatggttttcttttgggtgagaaccctaatgagaaaatctacattccattagatttggatttatgcatgatcggttcttggccaagagattccttgtatattctgccaaatggacatccctcatttgcattgctgtgtgcattaaggctttggacaactcccagaaatcgtcggaaagccttaagtcatcagatctactctggatcattgttatctgttgaaaatgagctggagattctgaaatggttggtcaagaagtgcaaggaaactttgcagcaattacctacgactatagaatttgatgataatttgctcgtccttttatgcaagctacagaacagcactagctgcataacagaaatgaaccggtcaatctttgagcaagaatttgctccattcttcagatttcatggcttcaagctggattgttctatacacagtaaactcccagttcgtcttctacgatccctggaaagatggggattagctgtccagtggagatgcaactacaaaagaaccctaaccaagtgtattgtgcactgtaaaagtttagttcacgagctttcgttgcaacaaaatcaacaatag

SEQ ID NO: 3 - DNA, 2001 bp, Oryza sativa
aagggatagtttggtcctaaaatttttgcaaatattttgaagaggtagctagtggcatatctctactacttaaaaaataaaagtgcttccattgtccgttaaaaaaaccgggcgaaaaaaccgaatctatctgatccaaaaaaaccgggccaaaaaaaccggaaaaagaaaagaaaaaccaaacgtcaataaaaaccagtcgttaaaaaatgtccgatcaaaaaaaacgggtgaaaaaaaaactgaaaaaagaaaagaaaaccgaatcggttcgatccaaaaaaaaagggcgaaaataagtgaaaagaaaaacgaatccgactctgtcaatgcggtaaaaagagggtgaaaaaaatatgtccgattccaaactctgcattggtgaaaaaaattgatgtgccggattgaagatctttttgcaaaaacggaacatacgtgtcgagagcattccatttttatataatttttatttttagtttgtacttttgcatttaagtccctgtaatttttatatttacatttaagtccctataatcttgcaataagatacttgaggtcgtttttgttaaaaaaataataaaagagagagaacaaaggcagaaaggtgcattaaaagaaaaataaaagctgcacaaaaaagaaaataaagaaaaacaacaacaacaacaaaagtctttttcccctaagtggcgaaaaatactgtagatccgaacggaaaaaaaatctgatttatataaaaggcaaaaaaaaagtggtaaaaaaaatgctagatccgattcctttataaacggaaaaaaagtctgattcctataaaggccaaaaaaattctaaaaaaatatctaaaagagttcggccgattccctaaaaaaatggtgaaacaaaatccaatttctaaccagtatatatctcttctctatctctaatctaatctaactattttaaaagagaaaatgtacgagaaaataaaacggttcaaaaaaaatgtgtgaggcaaaagtcagaaaaaaacaccaaaaaaacatgctaaaaatgttttccatctttaataaaaaaaataaaaaatgcgcgagaaaaattatttctatcgtcggtaaaatattttttaaaaaaacatgcgaggaaaaacctgtctgaaataacgcgtcatttctaaaaaaatggttttaaaaaaccgcacaagaaaaataacaccgtctactaaaaacaaattgctttaaaaaaattgtcagaaaaaagataaattgatggacgcttgaatcgaataggatcattgattttttgccaactactatatggctttatttcgtcatatattctcttgtattggaaaaaagagaaaaaaagcattcgaaaaaaacaagcaaaaacaacatgcaagaaaacaggcaaaaaaacacacgagaaaagaaaagcaaaaaaaggagaaaaatatagttttcttcatcagtaaaaaaaagaaaaaaacatgctaggaaaaaacttttagaaagaaatgcgccatttctgaaaaatggtttgagaaaatcgcgcaaggaaaaaaatacactgtttataaaaaacaagccgcttattaaaatagaaacattgtcattgacatgattatgcatgaaaaacatatattatcattagaatttttttacccgttacaatgcacaaacatttttgctagtaacaaataagtgtgaaattataatggtataaattcaaatagctgaatagtaatgatatatttcaaaaccgtaaaaattatagtggcataacccatacaaaatacttgctgtactagttgctatggttgtacagctatgtttggtttcgaaactactaatgataatcagaattttcaaagtttaactaaatcttatccaaatatcgaatatttataactgtagagtacttcatatataacacatattagagttatcaaaaaaaattcaaggtggaaattacagtagcagagtagagccaacaacaaaataattcagcttggacaattagtag

SEQ ID NO: 4 - DNA, 17 bp, artificial sequence
  primer
cctcgacggcgatgtgg

SEQ ID NO: 5 - DNA, 20 bp, artificial sequence
  primer
aaggggtcttgtccttgtca

SEQ ID NO: 6 - DNA, 20 bp, artificial sequence
  primer
ccacgggttcaccaacttga

SEQ ID NO: 7 - DNA, 23 bp, artificial sequence
  primer
ccaaagttcgacttggaatgaca

SEQ ID NO: 8 - DNA, 20 bp, artificial sequence
  primer
gtttccatgagcaccttcgt

SEQ ID NO: 9 - DNA, 20 bp, artificial sequence
  primer
ctcaagcaagaactgcatcg

SEQ ID NO: 10 - DNA, 20 bp, artificial sequence
  primer
tgccgtaacctgctcatgta

SEQ ID NO: 11 - DNA, 20 bp, artificial sequence
  primer
gagccctgaagccatttgta

SEQ ID NO: 12 - DNA, 20 bp, artificial sequence
  primer
ttgactttggcagcagtgac

SEQ ID NO: 13 - DNA, 20 bp, artificial sequence
  primer
catattcaatggcgcagatg

SEQ ID NO: 14 - DNA, 20 bp, artificial sequence
  primer
ggagcacgaagaagtccaag

SEQ ID NO: 15 - DNA, 20 bp, artificial sequence
  primer
cctccccatgcattgtaatc

SEQ ID NO: 16 - DNA, 20 bp, artificial sequence
  primer
gctcgtccttttatgcaagc

SEQ ID NO: 17 - DNA, 20 bp, artificial sequence
  primer
ccatctttccagggatcgta

SEQ ID NO: 18 - DNA, 20 bp, artificial sequence
  primer
cggtgttctagcgaaacaga

SEQ ID NO: 19 - DNA, 20 bp, artificial sequence
  primer
ccgtatgttccatcatgtgc

SEQ ID NO: 20 - DNA, 20 bp, artificial sequence
  primer
caatgctgatagcggtgaga

SEQ ID NO: 21 - DNA, 20 bp, artificial sequence
  primer
gtggccatacctcgcatagt

SEQ ID NO: 22 - DNA, 20 bp, artificial sequence
  primer
gtgagcgtccctctcctatg

SEQ ID NO: 23 - DNA, 20 bp, artificial sequence
  primer
tctcttcctcctcaggctca

SEQ ID NO: 24 - DNA, 20 bp, artificial sequence
  primer
atgaagatgaaccggaaacg

SEQ ID NO: 25 - DNA, 20 bp, artificial sequence
  primer
gccaaagatacctccgtctg

SEQ ID NO: 26 - DNA, 20 bp, artificial sequence
  primer
accattggtgctgagcgttt

SEQ ID NO: 27 - DNA, 21 bp, artificial sequence
  primer
cgcagcttccattcctatgaa

SEQ ID NO: 28 - DNA, 23 bp, artificial sequence
  primer
agctgcgccgatggtttctacaa

SEQ ID NO: 29 - DNA, 21 bp, artificial sequence
  primer
atcgcctcgctccagtcaatg

SEQ ID NO: 30 - DNA, 20 bp, artificial sequence
  small RNA
tctttgagcaagaatttgct

SEQ ID NO: 31 - protein, 483 aa, Zea mays
MEALLKWAAELGVSDSPSPPSSSPSGNISSPSCLGGSLVVADFPDAGGRGLAAARDLRRGELVLRLPRAALLTSDRVTADDPRIAACVSAHKPRLSSVQILIVCLLAEVGKGSNSVWYPYLCQLPSYYTILATFNDFEVEALQVDDAIWVAQKAKSAIKSDWEDATPLMKELEFKPKLLMFKSWLWAFATVSSRTLHIAWDEAGCLCPVGDLFNYAAPDDDTLLEDEDTAELTNYQQKNGMTNSSERLTDGGYEDCNAYCLYARKNYKKGEQVLLAYGTYTNLELLEHYGFLLGENPNEKTFIELDLDICSVGTWPKDSMYIHPNGHPSFALLCALRLWSTPTNRRKAVSHQVYSGSMLSTENEMGIMKWLINKCEGTLQQLPTTVEFDDSLLVLLRTIQNSSNCRADAKRSGFEQEFAVFLRFHRAEQDCSGNSLLPFRLMRSVERWELAVQWRCNYKKTLKKCISYCEGLVHELPLQLNQQ

SEQ ID NO: 32 - protein, 526 aa, Nicotiana tabacum
MDDEAEALNLKSFLRWATELGISDSPSTSITPSPASCLGYSLFVSNFPEAGGRGLAAARDIKKGELILRVPKGVLMTSQRLMRNDESLSIAVKKHASLCCTQILAVALLNEVNKGKSSRWWPYLKQFPRSYDTLADFGKFEIQALQIDDAIWAAQKASQKAEGEWKEASALMHDLKLKPQLLALKAWLWAFGSISSRTMHIPWDEAGCFCPVGDFFNYAAPGEETSNSEDQVTREAFSLQEDSMLNSVTELAAAHRLIDAGYEEDVSSYCFYARRNYQKGEQVLLSYGTYTNLELLQHYGFILSDNPNDKAFIPLEPNMYSLCSWESESLYIQPDGKPSFALLSTVRIWAVPQNNRRPVAHLVYSGYQLSIENEVVAMRWLAKKCRTILDILPTTAEEDGKLLVILDEFQETHQLVDIKEMPSALATELCAFMESKKIVSDGTCVISGVARRSIGRLKLAILWRYQYKKILSNCILHCTEVINDIISTKDLEYKEIFFKPANLHSHCFIPLHFRLLPCSVISSPTH

SEQ ID NO: 33 - protein, 491 aa, Arabidopsis thaliana
MDLEHQTMETFLRWAAEIGISDSIDSSRFRDSCLGHSLSVADFTHAGGRGLGAVRELKKGELVLKVPRKALMTTESIIAKDLKLSDAVNLHGSLSSTQILSVCLLYEMSKEKKSFWYPYLVHIPRDYDLLATFGEFEKQALQVEDAVWATEKAIAKCQSEWKEAGSLMKELELKPKFRSFQAWLWASATISSRTLHVPWDSAGCLCPVGDLFNYDAPGDYSNTPEGPESANNVEEAGLVVETHSERLTDGGFEEDVNAYCLYARRNYHLGEQVLLCYGTYTNLELLEHYGFMLEENSNDKVFIPLENSLFSLASSWPKDSLYIHQDGNPSFALISTLRLWLIPQSQRDKSVMRLVYAGSQISVKNEILVMKWMSEKCGSVLRDLPTSVTEDTVLLHNIDKLQDPELRLEQKETEAFGSEVRAFLDANCLWDVTVLSGKPIEFSRKTSRMLSKWRLSVQWRLSYKRTLADCISYCNEKMNNLLGTQDRIRDL

SEQ ID NO: 34 - DNA, 1452 bp, Zea mays
atggaggcgctcctcaagtgggcggccgagctgggcgtctccgactcgccgtcgcccccttcctcctccccctctggaaacatctcttcgccgtcctgcctcggcggctccctcgtcgtcgccgacttccccgacgccggcgggaggggcctcgcggcggcgcgtgacttgcggcgcggggagctggtgttgcgcctgccccgagccgcgctgctcacaagcgaccgcgtcacggccgacgaccccaggatcgccgcctgcgtcagcgcccacaagccccgcctctcctccgtgcagatactcatagtgtgcttattggccgaagtggggaaagggagcaactctgtctggtacccctacctgtgtcagctacccagctactacactatcttggctaccttcaatgattttgaagtcgaagctctccaagttgatgatgctatttgggttgcacaaaaggctaaaagtgccataaaatcagactgggaagatgcgacaccactaatgaaagaattggaatttaaacctaagcttttgatgtttaaatcctggctctgggcatttgcaacggtatcttctcgcacattacatattgcatgggatgaagctggttgcttatgccctgtgggtgatttgttcaattatgctgctcctgatgatgacactttattggaggacgaggatacagctgaacttacgaattaccagcagaagaatgggatgaccaattcatctgagagattaacagatggtggatacgaagattgtaatgcatactgtctgtatgcaagaaaaaattataaaaagggggagcaggtcctccttgcttatggaacatacacaaacctggaactccttgaacactatggctttcttttgggtgagaaccccaacgagaaaactttcatcgagttggatttggatatttgctcagtgggaacctggccaaaagattcaatgtatattcatccaaacggacacccctcatttgcattgttatgtgcattaaggctttggtcaactcccacaaaccgtagaaaggctgtcagtcatcaggtttactctggatcaatgctttcgactgaaaatgagatggggattatgaaatggttaatcaacaaatgtgaaggaactttgcaacaattacctacgacagtagaatttgatgacagtctgcttgtccttctacgcacaatacagaacagtagcaactgcagagcagatgcgaagcggtcaggttttgagcaggagtttgctgtgttcctccggttccatcgagcggagcaggattgttctggtaacagtctactcccttttcgacttatgagatctgtggaaagatgggaactagctgtccagtggagatgcaactacaagaagacgctgaagaaatgtatttcgtactgtgaaggtttagttcacgagcttcccttgcaactaaaccagcaataa

SEQ ID NO: 35 - DNA, 1581 bp, Nicotiana tabacum
atggatgatgaagctgaagctttgaacctgaagagcttcttgagatgggcaactgagcttggaatctcagattctccatcaacttctataaccccatcaccagcttcttgtttgggatactccctttttgtctctaatttccctgaagctggagggagaggtttagcagcagctcgtgatattaagaaaggggaattgattcttagagtcccaaagggagtgttaatgactagtcaacgtcttatgagaaacgatgagtcattgtcaattgctgtcaagaaacacgcttctctctgctgtactcagattttggctgttgcactattaaatgaagtgaacaaggggaagagttcacggtggtggccctatctaaagcagttcccccgcagttatgacacacttgcggattttggaaaattcgagatacaagcattgcaaattgatgatgccatctgggctgcacaaaaggcttctcagaaggctgaaggagagtggaaagaagctagtgcgcttatgcatgatctcaagctcaagccgcagctcctggctcttaaagcttggctatgggcctttggttctatatcctcgcgcaccatgcacataccttgggacgaagctggctgtttttgccctgtgggggatttcttcaactacgcggcacctggagaggaaacatctaattctgaagatcaggtcactagagaggcattttcattgcaagaagacagtatgcttaattcagtaactgaattggctgcagcacataggctaattgatgctggatatgaggaagacgtttcttcatactgcttctatgctagaagaaactaccagaaaggagaacaggttcttctaagctatggaacttacacaaatttggagcttcttcaacactacggatttattctttccgacaacccaaatgacaaggcctttatacctttagagccaaacatgtattctttgtgttcatgggagagcgagtcactctacattcagccagatgggaaaccatcctttgctctactatcaacagttcgaatctgggcagtccctcaaaacaatcgcaggccagttgcacacctagtctattcaggataccaactttcaatagagaacgaagttgttgcaatgagatggctagcgaagaaatgcagaactatactcgacattcttccaactacagctgaagaagacggtaagttgcttgtcatcctagatgaatttcaagaaactcatcaactcgtggatattaaagagatgccatcagcacttgcaactgagctttgtgctttcatggaaagcaagaagatagtaagcgacggaacttgtgtcatttcaggtgtagctagaaggtctattgggagattaaaattagcaatcctgtggaggtatcagtacaagaaaatacttagcaattgcattttacattgtactgaggtgattaatgatattatttctacaaaagatttagaatacaaggaaatcttttttaagcctgcgaatttacattcacattgctttattccattgcacttccgcctactaccttgttcagtaatttcttcaccaacacattaa

SEQ ID NO: 36 - DNA, 1476 bp, Arabidopsis thaliana
atggacttagaacaccaaaccatggagacgttcctgcgatgggcagcagagattggcatttcagattctattgattcttctcgatttcgcgattcgtgtcttggacattctctttccgtctccgacttccctgacgccggcgggagaggtttgggagctgctcgtgagctcaagaaaggagaattggttttgaaagtccccagaaaagccttgatgactacagaatccataattgctaaagatctaaaattgagcgatgctgttaatcttcacaattcactttcttcgactcagatactgagtgtatgcttattgtatgaaatgagcaaggagaagaagtctttttggtatccatacttgttccatatacctcgtgactatgatctcttggctacatttgggaatttcgagaagcaagctttacaagttgaagatgctgtttgggctacagaaaaagccacagccaagtgtcagtctgagtggaaagaagctggttcgttaatgaaggagttagagctgaagcctaagtttcggagtttccaagcatggctatgggcttctgcaacgatatcttcacgaacactgcatgtaccatgggatagtgctgggtgtttgtgtcctgtaggggacttgttcaactacgatgctcctggagattattcgaataccccacaaggtcctgagagtgctaataatgtagaggaagcaggacttgttgttgagacacactctgaaaggctcacagatggtggattcgaggaagatgtcaatgcttactgtctttatgcaagaaggaattatcagctaggagaacaggttcttctatgttatgggacttacacgaatctagagcttcttgagcactatgggtttatgttagaagagaactcaaatgacaaggtcttcattcctctagaaaccagcctattttctctagcttcttcatggcctaaagattctttatacattcatcaagacggtaagctgtcttttgcgctgatatccacgttgagactgtggctgatcccacagagccagcgtgacaagtcagttatgcgccttgtttacgctggatctcagatatctgtgaagaatgagattcttgtcatgaagtggatgtcagagaaatgtggaagtgttttaagggatctgccaacttctgtcacagaggacactgtgcttcttcataacattgacaaactccaagatccagaattgcgtttggagcagaaagaaacagaagcttttggcagcgaagtacgtgcttttcttgatgcaaattgtttgtgggatgtcactgtattatctgggaaacctatagaattttccaggaaaactagcaggatgctgagcaaatggagatggtctgtgcagtggaggctaagctacaagagaactctagcagattgcatctcttattgtaatgagaaaatgaataatctcttaggtacccaagataggcttagagatttgtaa

SEQ ID NO: 37 - DNA, 2001 bp, artificial sequence
  the promoter region
aagggatagtttggtcctaaaatttttgcaaatattttgaagaggtagctagtggcatatctctactacttaaaaaataaaagtgcttccattgtccgttaaaaaaaccgggcgaaaaaaccgaatctatctgatccaaaaaaaccgggccaaaaaaaccggaaaaagaaaagaaaaaccaaacgtcaataaaaaccagtcgttaaaaaatgtccgatcaaaaaaaacgggtgaaaaaaaaactgaaaaaagaaaagaaaaccgaatcggttcgatccaaaaaaaaagggcgaaaataagtgaaaagaaaaacgaatccgactctgtcaatgcggtaaaaagagggtgaaaaaaatatgtccgattccaaactctgcattggtgaaaaaaattgatgtgccggattgaagatctttttgcaaaaacggaacatacgtgtcgagagcattccatttttatataatttttatttttagtttgtacttttgcatttaagtccctgtaatttttatatttacatttaagtccctataatcttgcaataagatacttgaggtcgtttttgttaaaaaaataataaaagagagagaacaaaggcagaaaggtgcattaaaagaaaaataaaagctgcacaaaaaagaaaataaagaaaaacaacaacaacaacaaaagtctttttcccctaagtggcgaaaaatactgtagatccgaacggaaaaaaaatctgatttatataaaaggcaaaaaaaaagtggtaaaaaaaatgctagatccgattcctttataaacggaaaaaaagtctgattcctataaaggccaaaaaaattctaaaaaaatatctaaaagagttcggccgattccctaaaaaaatggtgaaacaaaatccaatttctaaccagtatatatctcttctctatctctaatctaatctaactattttaaaagagaaaatgtacgagaaaataaaacggttcaaaaaaaatgtgtgaggcaaaagtcagaaaaaaacaccaaaaaaacatgctaaaaatgttttccatctttaataaaaaaaataaaaaatgcgcgagaaaaattatttctatcgtcggtaaaatattttttaaaaaaacatgcgaggaaaaacctgtctgaaataacgcgtcatttctaaaaaaatggttttaaaaaaccgcacaagaaaaataacaccgtctactaaaaacaaattgctttaaaaaaattgtcagaaaaaagataaattgatggacgcttgaatcgaataggatcattgattttttgccaactactatatggctttatttcgtcatatattctcttgtattggaaaaaagagaaaaaaagcattcgaaaaaaacaagcaaaaacaacatgcaagaaaacaggcaaaaaaacacacgagaaaagaaaagcaaaaaaaggagaaaaatatagttttcttcatcagtaaaaaaaagaaaaaaacatgctaggaaaaaacttttagaaagaaatgcgccatttctgaaaaatggtttgagaaaatcgcgcaaggaaaaaaatacactgtttataaaaaacaagccgcttattaaaatagaaacattgtcattgacatgattatgcatgaaaaacatatattatcattagaatttttttacccgttacaatgcacaaacatttttgctagtaacaaataagtgtgaaattataatggtataaattcaaatagctgaatagtaacgatatatttcaaaaccgtaaaaattatagtggcataacccatacaaaatacttgctgtactagttgctatggttgtacagctatgtttggtttcgaaactactaatgataatcagaattttaaaagtttaactaaatcttatccaaatatcgaatatttataactgtagagtacttcatatataacacatattagagttatcaaaaaaaattcaaggtggaaattacagtagcagagtagagccaacaacaaaataattcagcttggacaattagtag